(19) United States
(12) Patent Application Publication
(10) Pub. No.: US 2002/0151563 A1
(43) Pub. Date: Oct. 17, 2002

(54) Farnesyl transferase inhibitors in combination with HMG CoA reductase inhibitors for the inhibition of abnormal cell growth

(75) Inventor: Shama M. Kajiji, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/103,251

(22) Filed: Mar. 21, 2002

Related U.S. Application Data
(60) Provisional application No. 60/279,965, filed on Mar. 29, 2001.

(51) Int. Cl. A61K 31/4709; A61K 31/55

(52) U.S. Cl. 514/312; 514/210.21; 514/217.07

(57) ABSTRACT

This invention relates to pharmaceutical compositions for the treatment of abnormal cell growth, such as cancer or benign hyperproliferative disorder, in a mammal, which comprises a therapeutically effective amount of a farnesyl transferase (FTase) inhibitor in combination with an hydroxymethylglutaryl coenzyme A (HMG CoA) reductase inhibitor and a pharmaceutically acceptable carrier.

### BACKGROUND OF THE INVENTION

This invention relates to pharmaceutical compositions for the treatment of abnormal cell growth in a mammal, which comprises a therapeutically effective amount of a farnesyl transferase (FTase) inhibitor and an hydroxymethylglutaryl coenzyme A (HMG CoA) reductase inhibitor, and a pharmaceutically acceptable carrier.

Oncogenes are genes that, when activated, encode protein components of signal transduction pathways which lead to the abnormal stimulation of cell growth and mitogenesis. Oncogene expression in cultured cells leads to cellular transformation, characterized by the ability of cells to grow in soft agar and the growth of cells as dense foci lacking the contact inhibition exhibited by non-transformed cells.

Mutation and/or overexpression of certain oncogenes is frequently associated with human cancers and other disorders involving abnormal (i.e., unregulated) cell growth. For example, the growth of benign and malignant tumors can be caused by the expression of an activated Ras oncogene or by activation of the Ras protein by another gene that has undergone oncogenic mutation. The abnormal growth of cells that occurs in the benign and malignant cells of other proliferative disorders can be caused by aberrant Ras activation. Mutated oncogenic forms of Ras are frequently found in many human cancers, most notably in more than 50% of colon and pancreatic carcinomas (Kohl et al., Science, Vol. 260, 1834 to 1837, 1993). The Ras oncogene is expressed in about 40% of solid malignant tumors that are unresponsive to conventional chemotherapies. The K-Ras isoform is expressed in about 90% of pancreatic tumors and about 40% of colorectal and lung cancers. The H-Ras isoform is expressed in about 40% of head and neck cancers. The N-Ras isoform is expressed in most thyroid cancers and about 25% of acute myeloid leukemias. To acquire the potential to transform normal cells into cancer cells or benign cells that exhibit abnormal growth, as defined below, the precursor of the Ras oncoprotein must undergo farnesylation of the cysteine residue located in a carboxyl-terminal tetrapeptide. Inhibitors of the enzyme that catalyzes this modification, farnesyl protein transferase, are therefore useful as anticancer agents for tumors in which Ras contributes to transformation.

The K-Ras isoform can be both farnesylated and geranyl-geranylated in intact cells. Potent inhibitors of the enzyme farnesyl (FTase) that are highly selective for FTase versus geranylgeranyl transferase I (GGTase I) can be incapable of blocking prenylation of mutant K- Ras and therefore ineffective at inhibiting growth of K-Ras expressing tumor cells.

The administration of a low dose HMG CoA reductase inhibitor in combination with a potent selective FTase inhibitor will block K-Ras prenylation and K-Ras function, as well as H-Ras prenylation and function. The activity of the protein prenyl transferases FTase and GGTase I is dependent on the concentrations of the isoprenoid substrates, farnesyl- and geranylgeranyl-pyrophosphates, respectively. Mevalonate is the first intermediate in the isoprenoid pathway, and its synthesis is dependent on the activity of HMG CoA reductase. Compounds such as lovastatin and compactin, which are tight binding inhibitors of HMG CoA reductase, block mevalonate formation and thus block the isoprenoid pathway. They therefore inhibit both FTase and GGTase I.

Japanese Patent Application JP7316076A, which was published on Dec. 5, 1995, refers to an anticancer pharmaceutical composition that contains limonene, which, while not a FTase inhibitor, has been shown to impair the incorporation of mevalonic acid-derived isoprene compounds into Ras and Ras related proteins, and pravastatin, which is an HMG CoA reductase inhibitor.

### SUMMARY OF THE INVENTION

The present invention relates to pharmaceutical compositions for the treatment of abnormal cell growth in a mammal, including a human, comprising a therapeutically effective amount of a FTase inhibitor and an HMG CoA reductase inhibitor and a pharmaceutically acceptable carrier, wherein the FTase inhibitor and the HMG CoA reductase inhibitor are present in amounts that render the composition effective in the treatment of abnormal cell growth.

In the pharmaceutical compositions of the present invention, the FTase inhibitor is selected from (a) compounds having the following formula 1:

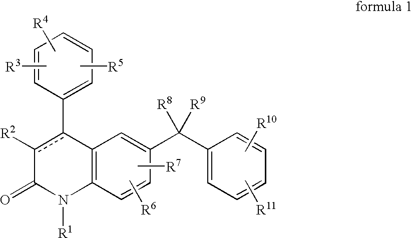

and the pharmaceutically acceptable salts, prodrugs and solvates thereof, wherein the dashed line indicates that the bond between C-3 and C-4 of the quinolin-2-one ring is a single or double bond;

R¹ is selected from H, C₁-C₁₀ alkyl, —(CR¹³R¹⁴)_{q}C(O)R¹², —(CR¹³R¹⁴)_{q}C(O)OR¹⁵, —(CR¹³R¹⁴)_{q}OR¹², —(CR¹³R¹⁴)_{q}SO₂R¹⁵, —(CR¹³R¹⁴)ₜ(C₃-C₁₀ cycloalkyl), —(CR¹³R¹⁴)ₜ(C₆-C₁₀ aryl), and —(CR¹³R¹⁴)ₜ(4-10 membered heterocyclic), wherein t is an integer from 0 to 5 and q is an integer from 1 to 5, said cycloalkyl, aryl and heterocyclic R¹ groups are optionally fused to a C₆-C₁₀ aryl group, a C₅-C₈ saturated cyclic group, or a 4-10 membered heterocyclic group; and the foregoing R¹ groups, except H but including any optional fused rings referred to above, are optionally substituted by 1 to 4 R⁶ groups;

R² is halo, cyano, —C(O)OR¹⁵, or a group selected from the substituents provided in the definition of R¹²;

each R³, R⁴, R⁵, R⁶, and R⁷ is independently selected from H, C₁-C₁₀ alkyl, C₂-C₁₀ alkenyl, halo, cyano, nitro, mercapto, trifluoromethyl, trifluoromethoxy, azido, —OR¹², —C(O)R¹², —C(O)OR¹², —NR¹³C(O)OR¹⁵, —OC(O)R¹², —NR¹³SO₂R¹⁵, —SO₂NR¹²R¹³, —NR¹³C(O)R¹², —C(O)NR¹²R¹³, —NR¹²R¹³, —CH=NOR¹², —S(O)ⱼR¹² wherein j is an integer from 0 to 2, —(CR¹³R¹⁴)ₜ(C₆-C₁₀ aryl), —(CR¹³R¹⁴),(4-10 membered heterocyclic), —(CR¹³R¹⁴)ₜ(C₃-C₁₀ cycloalkyl), and —(CR¹³R¹⁴)C=αCR¹⁶, and wherein in the foregoing R³, R⁴, R⁵, R⁶, and R⁷ groups t is an integer from 0 to 5, the cycloalkyl, aryl and heterocyclic moieties of the foregoing groups are optionally fused to a C₆-C₁₀ aryl group, a C₅-C₈ saturated cyclic group, or a 4-10 membered heterocyclic group; and said alkyl, alkenyl, cycloalkyl, aryl and heterocyclic groups are optionally substituted by 1 to 3 substituents independently selected from halo, cyano, nitro, trifluoromethyl, trifluoromethoxy, azido, —NR¹³SO₂R¹⁵, —SO₂NR¹²R¹³, —C(O)R¹², —C(O)OR¹², —OC(O)R¹²R^{13 NR}¹³C(O)RNR¹³C(O)R¹², —C(O)NR¹²R¹³, —NR¹²R¹³, OR¹², C₁-C₁₀ alkyl, C₂-C₁₀ alkenyl, C₂-C₁₀ alkynyl, -(CR¹³R¹⁴)ₜ(C₆-C₁₀ aryl), and —(CR¹³R¹⁴)ₜ(4-10 membered heterocyclic), wherein t is an integer from 0 to 5;

R⁹ is H, —OR¹², —NR¹²R¹³, —NR¹²C(O)R¹³, cyano, —C(O)OR¹³, —SR¹², —(CR¹³R¹⁴)ₜ(4-10 membered heterocyclic), wherein t is an integer from 0 to 5, or C₁-C₆ alkyl, wherein said heterocyclic and alkyl moieties are optionally substituted by 1 to 3 R⁶ substituents;

R⁹ is —(CR¹³R¹⁴),(imidazolyl) wherein t is an integer from 0 to 5 and said imidazolyl moiety is optionally substituted by 1 or 2 R⁶ substituents;

each R¹⁰ and R¹¹ is independently selected from the substituents provided in the definition of R⁶;

each R¹² is independently selected from H, C₁-C₁₀ alkyl, —(CR¹³R¹⁴)ₜ(C₃-C₁₀ cycloalkyl), —(CR¹³R¹⁴)ₜ(C₆-C₁₀ aryl), and —(CR¹³R¹⁴)ₜ(4-10 membered heterocyclic), wherein t is an integer from 0 to 5; said cycloalkyl, aryl and heterocyclic R¹² groups are optionally fused to a C₆-C₁₀ aryl group, a C₅-C₈ saturated cyclic group, or a 4-10 membered heterocyclic group; and the foregoing R¹² substituents, except H, are optionally substituted by 1 to 3 substituents independently selected from halo, cyano, nitro, trifluoromethyl, trifluoromethoxy, azido, —C(O)R¹³, —C(O)OR¹³, —OC(O)R¹³, —NR¹³C(O)R¹⁴, —C(O)NR¹³R¹⁴, —NR¹³R¹⁴, hydroxy, C₁-C₆ alkyl, and C₁-C₆ alkoxy;

each R¹³ and R¹⁴ is independently H or C₁-C₆ alkyl, and where R¹³ and R¹⁴ are as (CR¹³R¹⁴)_{q} or (CR¹³R¹⁴)ₜ each is independently defined for each iteration of q or t in excess of 1;

R¹⁵ is selected from the substituents provided in the definition of R¹² except R¹⁵ is not H;

R¹⁶ is selected from the list of substituents provided in the definition of R¹² and —SiR¹⁷R¹⁸R^{19;}

R¹⁷, R¹⁸ and R¹⁹ are each independently selected from the substituents provided in the definition of R¹² except R¹⁷, R¹⁸ and R¹⁹ are not H; and

provided that at least one of R³, R⁴ and R⁵ is —(CR¹³R¹⁴),C≡CR¹⁶ wherein t is an integer from 0 to 5 and R¹³, R¹⁴, and R¹⁶ are as defined above; and

(b) compounds of the formula 2 shown below:

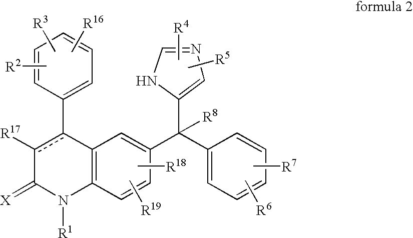

the pharmaceutically acceptable salts, prodrugs and solvates, wherein the dashed line indicates that the bond between C-3 and C-4 is a single or double bond;

X is oxygen or sulfur;

R¹ is hydrogen, C₁-C₁₂alkyl, Ar¹, Ar²C₁-C₆alkyl, quinolinylC₁-C₆ alkyl, pyridylC₁-C₆alkyl, hydroxyC₁-C₆alkyl, C₁-C₆alkyloxyC₁-C₆alkyl, mono- or di(C₁-C₆alkyl)aminoC₁-C₆alkyl, aminoC₁-C₆alkyl, or a radical of formula -Alk¹—C(═O)—R⁹, -Alk¹—S(O)—R⁹ or -Alk¹—S(O)₂—R⁹;

wherein Alk¹ is C₁-C₆alkanediyl;

R⁹ is hydroxy, C₁-C₆alkyl, C₁-C₆alkyloxy, amino, C₁-Calkylamino or C₁-Calkylamino substituted with C₁-C₆alkyloxycarbonyl;

R², R³ and R¹⁶ each independently are hydrogen, hydroxy, halo, cyano, C₁-C₆alkyl, C₁-C₆alkyloxy, hydroxyC,alkyloxy, C₁-C₆alkyloxyC₁-Cealkyloxy, aminoC₁-C₆alkyloxy, mono- or di(C₁-C₆alkyl)aminoC₁-C₆alkyloxy, Ar¹, Ar²C₁-C₆alkyl, Ar² oxy, Ar²C₁-C₆alkyloxy, hydroxycarbonyl, C₁-C₆alkyloxycarbonyl, trihalomethyl, trihalomethoxy, C₂-C₆alkenyl, or 4,4-dimethyloxazolyl; or when on adjacent positions R² and R³ taken together may form a bivalent radical of formula

—O—CH₂—O— (a-1),

—O—CH₂—CH₂—O— (a-2),

—O—CH═CH— (a-3),

—O—CH₂—CH₂— (a-4),

—O—CH₂—CH₂—CH₂— (a-5), or

—CH═CH—CH═CH— (a-6);

R⁴ and R⁵ each independently are hydrogen, halo, Ar¹, C₁-C₆alkyl, hydroxyC₁-C₆alkyl, C₁-CalkyloxyC₁-C₆alkyl, C₁-C₆alkyloxy, C₁-C₆alkylthio, amino, hydroxycarbonyl, C₁-C₆alkyloxycarbonyl, C₁-C₆alkylS(O)C₁-C₆alkyl or C₁₋₆alkylS(O)₂C₁-C₆alkyl;

R⁶ and R⁷ each independently are hydrogen, halo, cyano, C₁-C₆alkyl, C₁-C₆alkyloxy, Ar²oxy, trihalomethyl, C₁-C₆alkylthio, di(C₁-C₆alkyl)amino, or

when on adjacent positions R⁶ and R⁷ taken together may form a bivalent radical of formula

—O—CH₂—O— (c-1), or

—CH═CH—CH═CH— (c-2);

R⁸ is hydrogen, C₁₋₆alkyl, cyano, hydroxycarbonyl, C₁-C₆alkyloxycarbonyl, C₁-C₆alkylcarbonylC₁-C₆alkyl, cyanoC₁-C₆alkyl, C₁-C₆alkyloxycarbonylC₁-C₆alkyl, carboxyC₁-C₆alkyl, hydroxyC₁-C₆alkyl, aminoC₁-C₆alkyl, mono- or di(C₁-C₆alkyl)aminoC₁-C₆alkyl, imidazolyl, haloC₁-C₆ alkyl, C₁-C₆alkyloxyC₁-C₆alkyl, aminocarbonylC₁-C₆alkyl, or a radical of formula

—O—R¹⁰ (b-1),

—S—R¹⁰ (b-2),

—N—R¹¹R¹² (b-3),

wherein R¹⁰ is hydrogen, C₁-C₆alkyl, C₁-C₆alkylcarbonyl, Ar¹, Ar²C₁-C₆alkyl, C₁-C₆alkyloxycarbonylC₁-C₆alkyl, or a radical or formula -Alk²—OR¹³ or -Alk²—NR¹⁴R¹⁵;

R¹¹ is hydrogen, C₁-C₁₂alkyl, Ar¹ or Ar²C₁-C₆alkyl;

R¹² is hydrogen, C₁-C₆alkyl, C₁-C₁₆alkylcarbonyl, C₁-C₆alkyloxycarbonyl, C₁-C₆alkylaminocarbonyl, Ar¹, Ar²C₁-C₆alkyl, C₁-C₆alkylcarbonylC₁-C₆alkyl, a natural amino acid, Ar¹ carbonyl, Ar²C₁-C₆alkylcarbonyl, aminocarbonylcarbonyl, C₁-C₆alkyloxyC₁-C₆alkylcarbonyl, hydroxy, C₁-C₆alkyloxy, aminocarbonyl, di(C₁-C₆alkyl)aminoC₁-C₆alkylcarbonyl, amino, C₁-C₆alkylamino, C₁-C₆alkylcarbonylamino, or a radical of formula -Alk²—OR¹³ or -Alk²—NR¹⁴R¹⁵ wherein

Alk² is C₁-C₆alkanediyl;

R¹³ is hydrogen, C₁-C₆alkyl, C₁-C₆alkylcarbonyl, hydroxyC₁-C₆alkyl, Ar¹ or Ar²C₁-C₆alkyl;

R¹⁴ is hydrogen, C₁-C₆alkyl, Ar¹ or Ar²C₁-C₆alkyl;

R¹⁵ is hydrogen, C₁-C₆alkyl, C₁-C₆alkylcarbonyl, Ar¹ or Ar²C₁-C₆alkyl;

R¹⁷ is hydrogen, halo, cyano, C₁-C₆alkyl, C₁-C₆alkyloxycarbonyl, Ar¹;

R¹⁸ is hydrogen, C₁-C₆alkyl, C₁-C₆alkyloxy or halo;

R¹⁹ is hydrogen or C₁-C₆alkyl;

Ar¹ is phenyl or phenyl substituted with C₁-C₆alkyl, hydroxy, amino, C₁-C₆alkyloxy or halo; and

Ar² is phenyl or phenyl substituted with C₁-C₆alkyl, hydroxy, amino, C₁-C₆alkyloxy or halo.

R⁴ or R⁵ may also be bound to one of the nitrogen atoms in the imidazole ring. In that case the hydrogen on the nitrogen is replaced by R⁴ or R⁵ and the meaning of R⁴ and R⁵ when bound to the nitrogen is limited to hydrogen, Ar¹, C₁-C₆alkyl, hydroxyC₁-C₆alkyl, C₁-C₆alkyloxyC₁-C₆alkyl, C₁-C₆alkyloxycarbonyl, C₁-C₆alkylS(O)C₁-C₆alkyl, or C₁-C₆alkylS(O)₂C₁-C₆alkyl.

Preferred compounds of formula 1 include those wherein R¹ is H, C₁-C₆ alkyl, or cyclopropylmethyl; R² is H; R¹ is —C≡CR¹⁶; and R⁸ is —NR¹²R¹³, —OR¹², or a heterocyclic group selected from triazolyl, imidazolyl, pyrazolyl, and piperidinyl, wherein said heterocyclic group is optionally substituted by an R⁶ group. More preferred compounds include those wherein R⁹ is imidazolyl optionally substituted by C₁-C₆ alkyl; R³ is hydroxy, amino, or triazolyl; and R⁴, R⁵, R¹⁰ and R¹¹ are each independently selected from H and halo.

Other preferred compounds formula 1 include those wherein R¹ is —(CR¹³R¹⁴)ₜ(C₃-C₁₀ cycloalkyl) wherein t is an integer from 0 to 3; R² is H; R³ is —m C≡CR¹⁶; and R⁸ is —NR¹²R¹³, —OR¹², or a heterocyclic group selected from triazolyl, imidazolyl, pyrazolyl, and piperidinyl, wherein said heterocyclic group is optionally substituted by an R⁶ group. More preferred compounds include those wherein R⁹ is imidazolyl optionally substituted by C₁-C₆ alkyl; R⁸ is hydroxy, amino, or triazolyl; R⁴, R⁵, R¹⁰ and R¹¹ are each independently selected from H and halo; and R¹ is cyclopropylmethyl.

Other preferred compounds formula 1 include those wherein R³ is ethynyl and the other substituents are as defined above.

Specific preferred FTase inhibitors of the formula 1 include the following:

6-[(4-Chloro-phenyl)-hydroxy-(3-methyl-3H-imidazol-4-yl)-methyl]-4-(3-ethynyl-phenyl)-1-methyl-1H-quinolin-2-one (enantiomer A);

6-[(4-Chloro-phenyl)-hydroxy-(3-methyl-3H-imidazol-4-yl)-methyl]-4-(3-ethynyl-phenyl)-1-methyl-1H-quinolin-2-one (enantiomer B);

6-[Amino-(4-chloro-phenyl)-(3-methyl-3H-imidazol-4-yl)-methyl]-4-(3-ethynyl-phenyl)-1-methyl-1H-quinolin-2-one (enantiomer A);

6-[Amino-(4-chloro-phenyl)-(3-methyl-3H-imidazol-4-yl)-methyl]-4-(3-ethynyl-phenyl)-1-methyl-1H-quinolin-2-one (enantiomer B);

6-[(4-Chloro-phenyl)-hydroxy-(3-methyl-3H-imidazol-4-yl)-methyl]-4-(3-ethynyl-4-fluoro-phenyl)-1-methyl-1H-quinolin-2-one;

and the pharmaceutically acceptable salts, prodrugs and solvates of the foregoing compounds, as well as stereoisomers of the foregoing compounds.

Preferred compounds of formula 2 include compounds wherein the substituent R¹⁸ is situated on the 5 or 7 position of the quinolinone moiety and substituent R¹⁹ is situated on the 8 position when R¹⁸ is on the 7-position.

Other preferred compounds of formula 2 include compounds wherein X is oxygen.

Other preferred compounds of formula 2 include compounds wherein the dotted line represents a bond, so as to form a double bond.

Another preferred group of compounds of formula 2 are those compounds wherein R¹ is hydrogen, C₁-C₆alkyl, C₁-C₆alkyloxyC₁-C₆alkyl, di(C₁-C₆alkyl)aminoC₁-C₆alkyl, or a radical of formula -Alk¹—C(═O)R⁹, wherein Alk¹ is methylene and R⁹ is Cl, alkylamino substituted with C¹⁶alkyloxycarbonyl.

Still another preferred group of compounds of formula 2 includes those wherein R³ is hydrogen or halo; and R² is halo, C₁-C₆alkyl, C₂-C₆alkenyl, C₁-C₆alkyloxy, trihalomethoxy or hydroxyC₁-C₆alkyloxy.

A further group of preferred compounds of formula 2 include those wherein R² and R³ are on adjacent positions and taken together to form a bivalent radical of formula (a-1), (a-2) or (a-3).

A still further group of preferred compounds of formula 2 include those wherein R⁵ is hydrogen and R⁴ is hydrogen or C₁-C₆alkyl.

Yet another group of preferred compounds of formula 2 are those compounds wherein R⁷ is hydrogen; and R⁶ is C₁-C₆alkyl or halo, preferably chloro, especially 4-chloro.

A particular group of compounds of formula 2 are those wherein R⁸ is hydrogen, hydroxy, haloC₁-C₆alkyl, hydroxyC₁-Calkyl, cyanoC₁-C₆alkyl, C₁-C₆alkyloxycarbonylC₁-C,alkyl, imidazolyi, or a radical of formula —NR¹¹R¹² wherein R¹¹ is hydrogen or C₁-C₁₂alkyl and R¹² is hydrogen, C₁-C₆alkyl, C₁-C₆alkyloxy, hydroxy, C₁-C₆alkyloxyC₁-C₆alkylcarbonyl, or a radical of formula -Alk²—OR¹³ wherein R¹³ is hydrogen or C₁-C₆alkyl.

Preferred compounds of formula 1 are those compounds wherein R¹ is hydrogen, C₁-C₆alkyl, C₁-C₆alkyloxyC₁-C₆alkyl, di(C₁-C₆alkyl)aminoC₁-C₆alkyl, or a radical of formula -Alk¹—C(═O)—R⁹, wherein Alk¹ is methylene and R⁹ is C₁-Cealkylamino substituted with C₁-Calkyloxycarbonyl; R² is halo, C₁-C₆alkyl, C₂-C₆alkenyl, C₁-C₆alkyloxy, trihalomethoxy, hydroxyC₁-C₆alkyloxy or Ar¹; R³ is hydrogen; R⁴ is methyl bound to the nitrogen in 3-position of the imidazole; R⁵ is hydrogen; R⁶ is chloro; R⁷ is hydrogen; R⁸ is hydrogen, hydroxy, haloC₁-C₆alkyl, hydroxyC₁-C₆alkyl, cyanoC₁-C₆alkyl, C₁-C₆alkyloxycarbonylC₁-C₆alkyl, imidazolyl, or a radical of formula —NR¹¹R¹² wherein R¹¹ is hydrogen or C₁-C₁₂alkyl and R¹² is hydrogen, C₁-C₆alkyl, C₁-C₆alkyloxy, C₁-C₆alkyloxyC₁-C₆alkylcarbonyl, or a radical of formula -Alk²—OR¹³ herein R¹³ is C₁-C₆alkyl; R¹⁷ is hydrogen and R¹⁸ is hydrogen.

Preferred FTase inhibitors of formula 2 are the following:

4-(3-chlorophenyl)-6-[(4-chlorophenyl)hydroxy(1-methyl-1H-imidazol-5-yl)methyl]-1-methyl-2(1H)-quinolinone,

6-[amino(4-chlorophenyl)(1-methyl-1H-imidazol-5-yl)methyl]-4-(3-chlorophenyl)-1-methyl-2(1H)-quinolinone (enantiomer A);

6-[amino(4-chlorophenyl)(1-methyl-1H-imidazol-5-yl)methyl]-4-(3-chlorophenyl)-1-methyl-2(1H)-quinolinone (enantiomer B);

6-[(4-chlorophenyl)hydroxy(1-methyl-1H-imidazol-5-yl)methyl]-4-(3-ethoxyphenyl)-1-methyl-2(1H)-quinolinone;

6-[amino(4-chlorophenyl)(1-methyl-1H-imidazol-5-yl)methyl]-4-(3-ethoxyphenyl)-1-methyl-2(1H)-quinolinone,

6-amino(4-chlorophenyl)(1-methyl-1H-imidazol-5-yl)methyl]-1-methyl-4-(3-propylphenyl)-2(1H)-quinolinone;

and the pharmaceutically acceptable salts, prodrugs and solvates of the foregoing compounds.

One preferred FTase salt of the present invention is 6-[(4-chlorophenyl)(1-methyl-1H-imidazol-5-yl)methyl]-4-(3-ethoxyphenyl)-1-methyl-2(1H)-quinolinone monohydrochloride monohydrate.

In one embodiment of the present invention the HMG CoA reductase inhibitor is selected from the group consisting of atorvastatin, pravastatin, niacin, gemfibrozil, clofibrate, lovastatin, fluvastatin, simvastatin compactin and ZD4522 (AstraZeneca), and the pharmaceutically acceptable salts of the foregoing compounds. Preferably the HMG CoA reductase inhibitor is selected from the group consisting of atorvastatin, pravastatin, lovastatin, compactin, fluvastatin and simvastatin, and the pharmaceutically acceptable salts of the foregoing compounds. More preferably the HMG CoA reductase inhibitor is selected from the group consisting of atorvastatin, lovastatin, pravastatin and simvastatin and the pharmaceutically acceptable salts of the foregoing compounds. Most preferably the HMG CoA reductase inhibitor is atorvastatin or lovastatin and the pharmaceutically acceptable salts of the foregoing compounds.

This invention also relates to a pharmaceutical composition for inhibiting abnormal cell growth in a mammal which comprises a therapeutically effective amount of a FTase inhibitor and a HMG CoA reductase inhibitor, or a pharmaceutically acceptable salt or solvate or prodrug of the FTase inhibitor and the HMG CoA reductase inhibitor, in combination with an amount of a chemotherapeutic, wherein the amounts of the FTase inhibitor and the HMG CoA reductase inhibitor with the chemotherapeutic are effective in inhibiting abnormal cell growth.

Many chemotherapeutics are presently known in the art. In one embodiment, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, e.g. anti-androgens.

The invention also relates to a method of treating abnormal cell growth, comprising administerting to said mammal a pharmaceutical composition comprising a FTase inhibitor of formulas 1 or 2 and an HMG CoA reductase inhibitor as described above, wherein the FTase inhibitor and the HMG CoA reductase inhibitor are administered in amounts that render the combination of the two inhibitors effective in treating abnormal cell growth.

In one embodiment of the method of the present invention, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the para thyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In another embodiment of the present invention the abnormal cell growth is benign proliferative disorder.

This invention further relates to a method for inhibiting abnormal cell growth in a mammal which method comprises administering to the mammal an amount of a pharmaceutical composition of the present invention in combination with radiation therapy, wherein the amount of pharmaceutical composition in combination with the radiation therapy is effective in inhibiting abnormal cell growth in the mammal. Techniques for administering radiation therapy are known in the art, and these techniques can be used in the combination therapy described herein.

It is believed that the compositions of the present invention can render abnormal cells more sensitive to treatment with radiation for purposes of killing and/or inhibiting the growth of such cells. Accordingly, this invention further relates to a method for sensitizing abnormal cells in a mammal to treatment with radiation which comprises administering to the mammal an amount of a pharmaceutical composition of the present invention which is effective in sensitizing abnormal cells to treatment with radiation.

The invention also relates to a method for the treatment of abnormal cell growth in a mammal which comprises administering to said mammal a therapeutically effective amount of a compound of a FTase inhibitor and an HMG CoA reductase inhibitor, or a pharmaceutically acceptable salt or hydrate thereof, in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

This invention also relates to a method of and to a pharmaceutical composition for .w inhibiting abnormal cell growth in a mammal which comprises an amount of a compound of formula 1 or 2 and an HMG CoA reductase inhibitor, a pharmaceutically acceptable salt or solvate thereof, a prodrug thereof, or an isotopically-labelled derivative thereof, and an amount of one or more substances selected from anti-angiogenesis agents, signal transduction inhibitors, and antiproliferative agents.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of formula 1 or 2 and pharmaceutical compositions described herein. Examples of useful COX-II inhibitors include CELEBREX™ (celecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (ie. MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2 .1 ]octane-3-10 carboxylic acid hydroxyamide;

(2R, 3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hydroxycarbamoyl-cyclobutyl)-amino]-propionic acid;

4-[4-(4-chloro-phenoxy)-benzenesulfonylamino]-tetrahydro-pyran-4-carboxylic acid hydroxyamide;

(R) 3-[4-(4-chloro-phenoxy)-benzenesulfonylamino]-tetrahydro-pyran-3-carboxylic acid hydroxyamide;

(2R, 3R) 1-[4-(4-fluoro-2-methyl-benzyloxy)-benzenesulfonyl]-3-hydroxy-3-methyl-piperidine-2-carboxylic acid hydroxyamide;

3-[[4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesuffonyl]-(4-hydroxycarbamoyi-tetrahydro-pyran-4-yl)-amino]-propionic acid;

3-exo-3-[4-(4-chloro-phenoxy)-benzenesulfonylamino]-8-oxa-bicyclo[3.2.1 ]octane-3-carboxylic acid hydroxyamide;

3-endo-3-[4-(4-fluoro-phenoxy)-benzenesulfonylamino]-8-oxa-bicyclo[3.2.1 ]octane-3-3-carboxylic acid hydroxyamide; and

(R) 3-[4-(4-fluoro-phenoxy)-benzenesulfonylamino]-tetrahydro-furan-3-carboxylic acid hydroxyamide;

and pharmaceutically acceptable salts and solvates of said compounds.

Other anti-angiogenesis agents, including other COX-II inhibitors and other MMP inhibitors, can also be used in the present invention.

The compositions of the present invention can also be used with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.). These and other EGFR-inhibiting agents can be used in the present invention.

VEGF inhibitors, for example SU-5416 and SU-6668 (Sugen Inc. of South San Francisco, Calif., USA), can also be combined with the compound of the present invention. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO , 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.). These and other VEGF inhibitors can be used in the present invention as described herein.

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), can furthermore be combined with the compound of the invention, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued March 2, 1999), which are all hereby incorporated herein in their entireties by reference. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are incorporated in their entireties herein by reference. The erbB2 receptor inhibitor compounds and substance described in the aforementioned PCT applications, U.S. patents, and U.S. provisional applications, as well as other compounds and substances that inhibit the erbB2 receptor, can be used with the compound of the present invention in accordance with the present invention.

The compositions of the present invention can also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors, and the like. Specific CTLA4 antibodies that can be used in the present invention include those described in U.S. Provisional Application No. 60/113,647 (filed Dec. 23, 1998) which is incorporated by reference in its entirety, however other CTLA4 antibodies can be used in the present invention.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) expressing an activated Ras oncogene; (2) tumor cells in which the Ras protein is activated as a result of oncogenic mutation in another gene; (3) benign and malignant cells of other proliferative diseases in which aberrant Ras activation occurs; and (4) any tumors that proliferate by virtue of farnesyl protein transferase.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “halo”, as used herein, unless otherwise indicated, means fluoro, chloro, bromo or iodo. Preferred halo groups are fluoro, chloro and bromo.

The term “C₁-C₆alkanediyl”, as used herein, unless otherwise indicated, means bivalent straight and branched chained saturated hydrocarbon radicals having from 1 to 6 carbon atoms, such as, for example, methylene, 1,2-ethanediyl, 1,3-propanediyl, 1,4-butanediyl, 1,5-pentanediyl, 1,6-hexanediyl and the branched isomers thereof.

The term “alkyl”, as used herein, unless otherwise indicated, includes saturated monovalent hydrocarbon radicals having straight or branched moieties, such as, for example, methyl, ethyl, propyl, butyl, pentyl, hexyl and the like.

The term “cycloalkyl”, as used herein, unless otherwise indicated, includes cyclic alkyl moieties wherein alkyl is as defined above.

The term “alkenyl”, as used herein, unless otherwise indicated, includes alkyl moieties having at least one carbon-carbon double bond wherein alkyl is as defined above. Examples of alkenyl include ethenyl, 2-propenyl, 3-butenyl, 2-pentenyl, 3-pentenyl, 3-methyl-2-butenyl, and the like.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O—alkyl groups wherein alkyl is as defined above.

The term “C(═O)” refers to a carbonyl group, “S(O)” refers to a sulfoxide and “S(O)₂” to a sulfon.

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one or more heteroatoms, generally 1 to 4 heteroatoms, each selected from O, S and N, wherein each heterocyclic group has from 4-10 atoms in its ring system. Non-aromatic heterocyclic groups include groups having only 4 atoms in their ring system, but aromatic heterocyclic groups must have at least 5 atoms in their ring system. The heterocyclic groups include benzo-fused ring systems and ring systems substituted with one or more oxo moieties. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

In formula 1 wherein R¹³ and R¹⁴ are (CR¹³R¹⁴)q or (CR¹³R¹⁴), each is independently defined for each iteration of q or t in excess of 1. This means, for instance, that where q or t is 2 alkylene moieties of the type —CH₂CH(CH₃)—, and other asymmetrically branched groups, are included.

The term “pharmaceutically acceptable salt(s)”, as used herein, unless otherwise indicated, includes salts of acidic or basic groups that may be present in the compounds of formulas 1 and 2 or the HMG CoA reductase inhibitors. For example, pharmaceutically acceptable salts include sodium, calcium and potassium salts of carboxylic acid groups and hydrochloride sailts of amino groups. Other pharmaceutically acceptable salts of amino groups are hydrobromide, sulfate, hydrogen sulfate, phosphate, hydrogen phosphate, dihydrogen phosphate, acetate, succinate, citrate, tartrate, lactate, mandelate, methanesulfonate (mesylate) and p-toluenesulfonate (tosylate) salts. The preparation of such salts is described below.

The subject invention also includes isotopically-labelled compounds, and the pharmaceutically acceptable salts thereof, which are identical to those recited in formulas 1 and 2 but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶CI, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formulas 1 and 2 of this invention and prodrugs thereof can generally be prepared by carrying out the procedures disclosed in the Schemes and/or in the Examples and Preparations below, by substituting a readily available isotopically labelled reagent for a non-isotopically labelled reagent.

Compounds of formulas 1 and 2 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formulas 1 and 2. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, omithine and methionine sulfone. Examples of natural amino acids are glycine, alanine, valine, leucine, isoleucine, methionine, proline, phenylanaline, tryptophan, serine, threonine, cysteine, tyrosine, asparagine, glutamine, aspartic acid, glutamic acid, lysine, arginine, and histidine.

Additional types of prodrugs are also encompassed. For instance, free carboxyl groups can be derivatized as amides or alkyl esters. The amide and ester moieties may incorporate groups including but not limited to ether, amine and carboxylic acid functionalities. Free hydroxy groups may be derivatized using groups including but not limited to hemisuccinates, phosphate esters, dimethylaminoacetates, and phosphoryloxymethyloxycarbonyls, as outlined in D. Fleisher, R. Bong, B. H. Stewart, Advanced Drug Delivery Reviews (1996) 19, 115. Carbamate prodrugs of hydroxy and amino groups are also included, as are carbonate prodrugs and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in R. P. Robinson et al., J. Medicinal Chemistry (1996) 39, 10.

Certain compounds of formulas 1 and 2 may have asymmetric centers and therefore exist in different enantiomeric forms. All optical isomers and stereoisomers of the compounds of formulas 1 and 2, and mixtures thereof, are considered to be within the scope of the invention. With respect to the compounds of formulas 1 and 2, the invention includes the use of a racemate, one or more enantiomeric forms, one or more diastereomeric forms, or mixtures thereof. In particular, the carbon to which the R⁸ and R⁹ groups are attached represents a potential chiral center; the present invention encompasses all stereoisomers based on this chiral center. The compounds of formulas 1 and 2 may also exist as tautomers. This invention relates to the use of all such tautomers and mixtures thereof. Certain compounds of formula I may also include oxime moieties, such as where R³, R⁴, R⁵, R⁶ or R⁷ is —CH═NOR¹², that exist in E or Z configurations. The present invention includes racemic mixtures of compounds of formula 1 that include such oxime moieties or specific E or Z isomers of such compounds.

### DETAILED DESCRIPTION OF THE INVENTION

The present invention relates to pharmaceutical compositions for the treatment of abnormal cell growth in a mammal, including a human, comprising a therapeutically effective amount of a FTase inhibitor and an HMG CoA reductase inhibitor and a pharmaceutically acceptable carrier, wherein the FTase inhibitor and the HMG CoA reductase inhibitor are present in amounts that render the composition effective in the treatment of abnormal cell growth.

The HMG CoA reductase inhibitor is selected from the group consisting of atorvastatin, pravastatin, niacin, gemfibrozil, clofibrate, lovastatin, fluvastatin, simvastatin, compactin, and ZD4522 (AstraZeneca) and the pharmaceutically acceptable salts of the foregoing compounds. All of the aforementioned HMG CoA reductase inhibitors are commercially available.

The following references refer to compounds that exhibit activity as HMG CoA reductase inhibitors and which can be used, in combination with a FTase inhibitor, in the pharmaceutical compositions and methods of this invention, and to methods of preparing the same: U.S. Pat. No. 4,681,893, issued Jul. 21, 1987; U.S. Pat. No. 5,273,995, issued Dec. 28, 1993; U.S. Pat. No. 5,385,929, issued Jan. 31, 1995; U.S. Pat. No. 4,957,971, issued Sep. 18, 1990; U.S. Pat. No. 5,102,893, issued Apr. 7, 1992; U.S. Pat. No. 4,957,940, issued Sep. 18, 1990; U.S. Pat. No. 4,950,675, issued Aug. 21, 1990; U.S. Pat. No. 4,929,620, issued May 29, 1990; U.S. Pat. No. 4,923,861, issued May 8, 1990; U.S. Pat. No. 4,906,657, issued Mar. 6, 1990; U.S. Pat. No. 4,868,185, issued Sep. 19, 1989; U.S. Pat. No. 5,124,482 issued Jun. 23, 1992; U.S. Pat. No. 5,003,080, issued Mar. 26, 1991; U.S. Pat. No. 5,097,045, issued Mar. 17, 1992; U.S. Pat. No. 5,149,837, issued Sep. 22, 1992; U.S. Pat. No. 4,906,624, issued Mar. 6, 1990; U.S. Pat. No. 4,761,419, issued Aug. 2, 1988; U.S. Pat. No. 4,735,950, issued Apr. 5, 1988; U.S. Pat. No. 4,808,621, issued Feb. 28, 1989; U.S. Pat. No. 4,647,576, issued Mar. 3, 1987; U.S. Pat. No. 5,118,882, issued Jun. 2, 1992; U.S. Pat. No. 5,214,197, issued May 25, 1993; U.S. Pat. No. 5,321,046, issued Jun. 14, 1994; U.S. Pat. No. 5,260,440, issued Nov. 9, 1993; and U.S. Pat. No. 5,208,258 issued May 4, 1993; U.S. Pat. No. 5,369,125, issued Nov. 29, 1994; United States Patent H1345 issued Aug. 2, 1994; U.S. Pat. No. 5,262,435, issued Nov. 16, 1993; and U.S. Pat. No. 5,260,332, issued Nov. 9, 1993. Great Britian Patent Application GB 2,055,100, published Feb. 25, 1981; U.S. Pat. No. 4,499,289, issued Feb. 12, 1983; U.S. Pat. No. 4,645,854, issued Feb. 24, 1987; U.S. Pat. No. 4,613,610 issued Sep. 3, 1986; U.S. Pat. No. 4,668,699, issued May 26, 1987; U.S. Pat. No. 4,851,436, issued Jul. 25, 1989; U.S. Pat. No. 4,678,806, issued Jul. 7, 1987; U.S. Pat. No. 4,772,626, issued Sep. 20, 1988; U.S. Pat. No. 4,855,321, issued Aug. 8, 1989; European Patent Application EP 244364, published Nov. 4, 1987; U.S. Pat. No. 4,766,145, issued Aug. 23, 1988; U.S. Pat. No. 4,876,279, issued Oct. 24, 1989; U.S. Pat. No. 4,847,306, issued Jul. 11, 1989; U.S. Pat. No. 5,049,696, issued Sep. 17, 1991; European Patent Application EP 245,990, published Nov. 19, 1987; European Patent Application EP 251,625, published Jan. 7, 1988; U.S. Pat. No. 4,719,229, published Jan. 12, 1988; Japanese Patent Application 63014722, published Jan. 21, 1988; U.S. Pat. No. 4,736,064, issued Apr. 5, 1988; U.S. Pat. No. 4,738,982 issued Apr. 19, 1988; U.S. Pat. No. 4,845,237, issued Jul. 4, 1989; European Patent EP 306,263, granted Mar. 18, 1992; U.S. Pat. No. 5,026,708, issued Jun. 25, 1991; U.S. Pat. No. 4,863, 957, issued Sep. 5, 1989; U.S. Pat. No. 4,946,841, issued Aug. 7, 1990; European Patent 339358, granted Jul. 13, 1994; U.S. Pat. No. 4,937,264 issued Jun. 26, 1998; U.S. Pat. No. 4,876,366, issued Oct. 24, 1989; U.S. Pat. No. 4,921,974, issued May 1, 1990; U.S. Pat. No. 4,963,538 issued Oct. 16, 1990; U.S. Pat. No. 5,130,306, issued Jul. 14, 1992; U.S. Pat. No. 4,900,754 issued Feb. 13, 1990; U.S. Pat. No. 5,026,698, issued Jun. 25, 1991; U.S. Pat. No. 4,977,161, issued Dec. 11, 1990; U.S. Pat. No. 4,927,851, issued May 22, 1990; European Patent Application EP 373,507, published Jun. 20, 1990; U.S. Pat. No. 4,939,143, issued Jul. 3, 1990; U.S. Pat. No. 4,939,159, issued Jul. 3, 1990; U.S. Pat. No. 4,940,727, issued Jul. 10, 1990; U.S. Pat. No. 5,116,870, issued May 26, 1992; Australian Patent AU 635,545, granted Mar. 25, 1993; U.S. Pat. No. 5,098,391, issued Mar. 24, 1992; U.S. Pat. No. 5,294,724, issued Mar. 15, 1994; U.S. Pat. No. 5,001,255, issued Mar. 19, 1991; U.S. Pat. No. 5,149,834, issued Sep. 22, 1992; U.S. Pat. No. 5,089,523, issued Feb. 18, 1992; European Patent Application EP 465,265 published Jan. 8, 1992; U.S. Pat. No. 5,476,846, issued Dec. 19, 1995; U.S. Pat. No. 5,321,046, issued Jun. 14, 1994; U.S. Pat. No. 5,106,992, issued Apr. 21, 1992; U.S. Pat. No. 5,347,039, issued Sep. 13, 1994; Japanese Patent Application 4193836, published Jul. 13, 1992; Great Britian patent Application 2253787, published Sep. 23, 1992; U.S. Pat. No. 5,411,969, issued May 2, 1995; Japanese Patent Application 4,356,435, published Dec. 10, 1992; U.S. Pat. No. 5,266,707 issued Nov. 30, 1993; U.S. Pat. No. 5,455,247 issued Oct. 3, 1995; U.S. Pat. No. 5,475,029, issued Dec. 12, 1995; U.S. Pat. No. 5,591,772, issued Jan. 7, 1997; U.S. Pat. No. 5,286,746 issued Feb. 15, 1994; Japanese Patent Application JP 7089898, published Apr. 4, 1995; European Patent Application EP 677,039, published Oct. 18, 1995 and World Patent Application 96/08248, published Mar. 21, 1996.

The FTase inhibitor is selected from compounds having the formula 1 or 2 as defined above.

The FTase inhibitor compounds of formula 1 may be prepared as described in U.S. Pat. No. 6,150,377, the contents of which are hereby incorporated by reference. The FTase inhibitor compounds of formula 2 may be prepared as described in U.S. Pat. No. 6,037,350, the contents of which are hereby incorporated by reference. Other FTase inhibitor compounds may also be employed in the present invention such as those described in U.S. Pat. No. 5,968,952, the contents of which are hereby incorporated by reference.

The compounds of formulas 1 and 2 described above may have one or more stereogenic centers in their structure. Such stereogenic centers may be present in an R or an S configuration. Oxime moieties, such as where R³, R⁴, R⁵, R⁶ or R⁷ is —CH═NOR¹², may exist in E or Z configurations for formula 1.

The compounds of formulas 1 and 2 are generally racemic mixtures of enantiomers which can be separated from one another following resolution procedures familiar to those skilled in the art. The racemic compounds of formulas 1 and 2 may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of formulas 1 and 2 involves liquid chromatography using a chiral stationary phase. Said pure stereochemically isomeric forms may also be derived from the corresponding pure stereochemically isomeric forms of the appropriate starting materials, provided that the reaction occurs sterospecifically. Preferably if a specific stereoisomer is desired, said compound will be synthesized by stereospecfic methods of preparation. These methods will advantageously employ enantiomerically pure starting materials.

The compounds of formulas 1 and 2 that are basic in nature are capable of forming a wide variety of different salts with various inorganic and organic acids. Although such salts must be pharmaceutically acceptable for administration to animals, it is often desirable in practice to initially isolate the compound of formulas 1 or 2 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon evaporation of the solvent, the desired solid salt is readily obtained. The desired acid addition salt can also be precipitated from a solution of the free base in an organic solvent by adding to the solution an appropriate mineral or organic acid. Cationic salts of the compounds of formulas 1 and 2 are similarly prepared except through reaction of a carboxy group with an appropriate cationic salt reagent, such as sodium, potassium, calcium, magnesium, ammonium, N,N′-dibenzylethylenediamine, N-methylglucamine (meglumine), ethanolamine, tromethamine, or diethanolamine.

Patients that can be treated with a FTase inhibitor in combination with an HMG CoA reductase inhibitor according to the methods of this invention or using the pharmaceutical compositions of the invention include, for example, patients that have been diagnosed as having lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, gynecologic tumors (eg., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (eg., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphonas, cancer of the bladder, cancer of the kidney or ureter (eg, renal cell carcinoma, carcinoma of the renal pelvis), or neoplasms of the central nervous system (eg, primary CNS lymphona, spinal axis tumors, brain stem gliomas or pituitary adenomas).

Other more specific embodiments of this invention relate to any of the above pharmaceutical compositions and methods of treatment wherein the HMG CoA reductase inhibitor contained in such composition or used in such method is atorvastatin.

Other more specific embodiments of this invention relate to any of the above pharmaceutical compositions and methods of treatment wherein the HMG CoA reductase inhibitor contained in such composition or used in such method is lovastatin.

This invention relates both to methods of treating cancer in which the FTase inhibitor and the HMG CoA reductase inhibitor are administered together, as part of the same pharmaceutical composition, as well as to methods in which these two active agents are administered separately as part of an appropriate dose regimen designed to obtain the benefits of the combination therapy. The appropriate dose regimen, the amount of each dose administered, and specific intervals between doses of each active agent will depend upon the subject being treated, the type of cancer or abnormal cell growth and the severity of the condition. In carrying out the methods of this invention, the FTase inhibitor will be administered in the amounts disclosed in the literature, or otherwise believed to be effective, for the administration of such compound as a single active agent for the treatment of cancer or the inhibition of abnormal cell growth, and the HMG CoA reductase inhibitor will be administered in an amount that is about one quarter to one half of the amount disclosed in the literature, or otherwise believed to be effective, for administration of such compound as a single agent for the treatment of hypercholesterolemia.

Administration of the compounds of the present invention (hereinafter the “active compounds”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compounds (i.e., the Ftase inhibitor and the HMG CoA reductase inhibitor) administered will be dependent on the subject being treated, the severity of the disorder or condition, the rate of administration and the judgement of the prescribing physician. However, an effective dosage is in the range of about 0.001 to about 100 mg per kg body weight per day, preferably about 1 to about 35 mglkg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compounds may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl4-oxoquinazolin-6-ylmethyl)-N- methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of active compounds in either sesame or peanut oil or in aqueous propylene glycol may be employed. The aqueous solutions should be suitably buffered if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The pharmaceutical compositions may also be administered to a mammal other than a human. The dosage to be administered to a mammal will depend on the animal species and the disease or disorder being treated. The pharmaceutical compositions may be administered to animals in the form of a capsule, bolus, tablet or liquid drench. The pharmaceutical compositions may also be administered to animals by injection or as an implant. Such formulations are prepared in a conventional manner in accordance with standard veterinary practice. As an alternative the pharmaceutical compositions may be administered with the animal feedstuff and for this purpose a concentrated feed additive or premix may be prepared for mixing with the normal animal feed.

The activity of compounds as FTase inhibitors may be determined by their ability, relative to a control, to inhibit Ftase in vitro. This procedure is described below.

A crude preparation of human farnesyl transferase (FTase) comprising the cytosolic fraction of homogenized brain tissue was used for screening compounds in a 96-well assay format. The cytosolic fraction was prepared by homogenizing approximately 40 grams fresh tissue in 100 ml of sucrose/MgCl₂/EDTA buffer (using a Dounce homogenizer; 10-15 strokes), centrifuging the homogenates at 1000 ×g for 10 minutes at 4° C., re-centrifuging the supernatant at 17,000 ×g for 15 minutes at 4° C., and then collecting the resulting supernatant. This supernatant was diluted to contain a final concentration of 50 mM Tris HCI (pH 7.5), 5 mM DTT, 0.2 M KCI, 20 μM ZnCl₂, 1 mM PMSF and re-centrifuged at 178,000 ×g for 90 minutes at 4° C. The supernatant, termed “crude FTase” was assayed for protein concentration, aliquoted, and stored at −70° C.

The assay used to measure in vitro inhibition of human FTase is a modification of the method described by Amersham LifeScience for using their Farnesyl transferase (3H) Scintillation Proximity Assay (SPA) kit (TRKQ 7010). FTase enzyme activity was determined in a volume of 100 μL containing 50 mM N-(2-hydroxy ethyl) piperazine-N′-(2-ethane sulfonic acid) (HEPES), pH 7.5, 30 mM MgCl₂, 20 mM KCI, 25 mM Na₂HPO₄, 5 mM dithiothreitol (DTT), 0.01% Triton X-100, 5% dimethyl sulfoxide (DMSO), 20 μg of crude FTase, 0.12 pM [3H]-farnesyl pyrophosphate ([3H]-FPP; 36000 dpm/pmole, Amersham LifeScience), and 0.2 μM of biotinylated Ras peptide KTKCVIS (Bt-KTKCVIS obtained from AnaSpec, Inc., San Jose, Calif.) that is N-terminally biotinylated at its alpha amino group. The reaction was initiated by addition of the enzyme and terminated by addition of EDTA (supplied as the STOP reagent in kit TRKQ 7010) following a 45 minute incubation at 37° C. Prenylated and unprenylated Bt-KTKCVIS is captured by adding 10 μL of steptavidin-coated SPA beads (RPNQ0007) per well and incubating the reaction mixture for 30 minutes at room temperature. The amount of radioactivity bound to the SPA beads was determined using a MicroBeta 1450 plate counter. Under these assay conditions, the enzyme activity was linear with respect to the concentrations of the prenyl group acceptor, Bt-KTKCVIS, and crude FTase, and inhibition of Bt-KTKCVIS interaction with FTase can be detected. The enzyme activity was saturated with respect to the prenyl donor, FPP. The assay reaction time was also in the linear range.

The test compounds were routinely dissolved in 100% DMSO. Inhibition of farnesyl transferase activity was determined by calculating percent incorporation of tritiated-farnesyl in the presence of the test compound versus its incorporation in control wells (absence of inhibitor). An IC50 value, that is, the concentration required to produce half maximal farnesylation of Bt-KTKCVIS, was determined for each compound from the dose-responses obtained.

A fluorsecence assay for FTase activity that can be used to screen for FTase inhibitors is described in UK Patent Application GB 2,267,966, which was published on Dec. 22, 1993.

The activity of compounds as HMG CoA reductase inhibitors may be determined by the procedure described by Dugan et al, Achiv. Biochem. Biophys., (1972), 152, 21-27. In this method, the level of HMG-CoA enzyme activity in standard laboratory rats is increased by feeding the rats a chow diet confining 5% cholestyramine for four days, after which the rats are sacrificed. The rat livers are homogenized, and the incorporation of cholesterol-¹⁴C-acetate into nonsaponifiable lipid by the rat liver homogenate is measured. The micromolar concentration of compound required for 50% inhibition of sterol synthesis over a one-hour period is measured, and expressed as an IC₅₀ value.

A second method (designated COR screen) is that described by T. Kita, et al, J. Clin. Invest., (1980), 66: 1094-1100. In this method, the amount of ¹⁴C-HMG-CoA converted to ¹⁴C-mevalonate in the presence of a purified enzyme preparation of HMG-CoA reductase is measured. The micromolar concentration of compound required for 50% inhibition of cholesterol synthesis is measured and recorded as an IC₅₀ value.

The various methods of this invention may be practiced as part of a therapy that includes the administration of one or more other anti-tumor substances, for example, those selected from mitotic inhibitors, for example, vinblastine; alkylating agents, for example, cisplatin, carboplatin and cyclophosphamide; antimetabolites, for example, 5-fluorouracil, cystosine arabinoside and hydroxyurea, or, for example, one of the preferred antimetabolites disclosed in European Patent Application No. 239362 such as N-{5-[N-(3,4-dihydro-2-methyl-4-oxoquinazolin-6-ylmethyl)-N-methylamino]-2-thenoyl}-L-glutamic acid; intercalating antibiotics, for example, adriamycin and bleomycin; enzymes, for example, asparaginase; topoisomerase inhibitors, for example, etoposide; biological response modifiers, for example, interferon; and anti-hormones, for example, antioestrogens such as ‘NOLVADEX’ (tamoxifen) or antiandrogens such as ‘CASODEX’ (4′-cyano-3-(4-fluorophenylsulphonyl)-2-hydroxy-2-methyl-3′-10 (trifluoromethyl)propionanilide. Such therapies may be achieved by way of the simultaneous, sequential or separate dosing of the individual components of the therapy. According to this aspect of the invention, there is provided a pharmaceutical product comprising a pharmaceutically acceptable carrier, as described above, one or both of an HMG CoA reductase inhibitor and a FTase inhibitor, and an additional anti-tumor agent, as described above.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The example provided below illustrates the present invention. It is to be understood that the scope of the present invention is not limited in any way by the scope of the following example.

The effectiveness of the FTase inhibitor 6-[(4-chloro-phenyl)-hydroxy-(3-methyl-3H-imidazol4-yl)-methyl]-4-(3-ethynyl-phenyl)-1-methyl-1H-quinolin-2-one on prenylation of K-ras 4B in intact cells is enhanced by a minimally effective dose of lovastatin. Semi-confluent monolayers of the NIH-3T3 tranfectant overexpressing mutant K-ras 4B are treated for 72 hours at 37° C. with increasing concentrations (0, 1.0 μm, 5.0 μm 10.0 μm and 25 μm) of 6-[(4-chloro-phenyl)-hydroxy-(3-methyl-3H-imidazol4-yl)-methyl]4-(3-ethynyl-phenyl)-1-methyl-1H-quinolin-2-one in the presence and absence of 5,M of hydrolysed lovastatin. Cells are lysed in a RIPA lysis buffer (50 mM tris[hydroxymethyl]amino-methane, 0.15M sodium chloride, 1% sodium deoxycholate, 1% Triton X-100, 0.1% SDS, 0.25 sodium azide; pH 8.5) containing 1 mM of DTT (dithiothreitol; Boehringer Mannheim, Indianapolis, Ind.) and protease inhibitors (Aprotinin, Leupeptin, Antipain, Pefabloc at final concentrations of 10 μg/ml, 2 μg/ml, 2 μg/ml and 50 μM, respectively; Boehringer Mannheim, Indianapolis, Ind.) and boiled for 3 minutes. Equal amounts of protein (100 igalane) are resolved by SDS-PAGE on 12.5% gels and transferred to Immobilon-P membranes (Intergrated Separation Systems, Natick, Mass.). The membranes are immunoblotted for 1 hr with 2.5 μg/ml of anti-Pan-ras (Ab-3) monoclonal antibody (Calbiochem, La Jolla, Calif.). The blots are incubated with peroxidase-conjugated secondary antibody, and the immunoblotted Ras protein are detected by enhanced chemiluminescence (Amersham Life Products, Arlington Heights, Ill.). Percent of prenylated Ras is determined by densitometric scanning using MasterScan 3.0 (Scanalytics, Billerica, Mass.). The effectiveness of the FTase inhibitor, 6-[(4-Chloro-phenyl)-hydroxy-(3-methyl-3H-imidazol4-yl)-methyl]-4-(3-ethynyl-phenyl)-1-methyl-1H-quinolin-2-one on the inhibition of prenylation of K-ras 4B in intact cells is enhanced by the HMG CoA reductase inhibitor, lovastatin.

1. A pharmaceutical composition for treating abnormal cell growth, comprising a therapeutically effective amount of a FTase inhibitor and an HMG CoA reductase inhibitor and a pharmaceutically acceptable carrier, wherein said FTase inhibitor is selected from: from (a) compounds having the following formula 1: 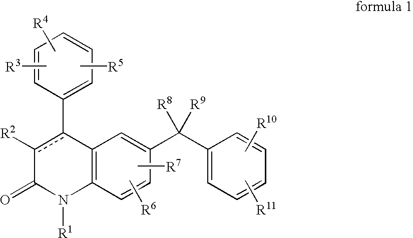
and the pharmaceutically acceptable salts, prodrugs and solvates thereof, wherein the dashed line indicates that the bond between C-3 and C-4 of the quinolin-2-one ring is a single or double bond;
R¹ is selected from H, C₁-C₁₀ alkyl, —(CR¹³R¹⁴)_{q}C(O)R¹², —(CR¹³R¹⁴)_{q}C(O)OR¹⁵, —(CR¹³R¹⁴)_{q}OR¹²—(CR¹³R¹⁴)_{q}SO₂R¹⁵, —(CR¹³R¹⁴)ₜ(C₃-C₁₀ cycloalkyl), —(CR¹³R¹⁴)ₜ(C₆-C₁₀ aryl), and —(CR¹³R¹⁴)ₜ(4-10 membered heterocyclic), wherein t is an integer from 0 to 5 and q is an integer from 1 to 5, said cycloalkyl, aryl and heterocyclic R¹ groups are optionally fused to a C₆-C₁₀ aryl group, a C₆-C₈ saturated cyclic group, or a 4-10 membered heterocyclic group; and the foregoing R¹ groups, except H but including any optional fused rings referred to above, are optionally substituted by 1 to 4 R⁶ groups;
R² is halo, cyano, —C(O)OR¹⁵, or a group selected from the substituents provided in the definition of R¹²;
each R³, R⁴, R⁵, R⁶, and R⁷ is independently selected from H, C₁-C₁₀ alkyl, C₂-C₁₀ alkenyl, halo, cyano, nitro, mercapto, trifluoromethyl, trifluoromethoxy, azido, —OR¹², —C(O)R¹², —C(O)OR¹², —NR¹³C(O)OR¹⁵, —OC(O)R¹², —NR¹³SO₂R¹⁵, —SO₂NR¹²R¹³, —NR¹³C(O)R¹², —C(O)NR¹²R¹³, —NR¹²R¹³, —CH═NOR¹², —S(O)ⱼR¹² wherein j is an integer from 0 to 2, —(CR¹³R¹⁴)(C₆-C₁₀ aryl), —(CR¹³R¹⁴),(4-10 membered heterocyclic), —(CR¹³R¹⁴)ₜ(C₃-C₁₀cycloalkyl), and —(CR¹³R¹⁴)ₜC≡CR¹⁶, and wherein in the foregoing R³, R⁴, R⁵, R⁶, and R⁷ groups t is an integer from 0 to 5, the cycloalkyl, aryl and heterocyclic moieties of the foregoing groups are optionally fused to a C₆-C₁₀ aryl group, a C₅-C₈ saturated cyclic group, or a 4-10 membered heterocyclic group; and said alkyl, alkenyl, cycloalkyl, aryl and heterocyclic groups are optionally substituted by 1 to 3 substituents independently selected from halo, cyano, nitro, trifluoromethyl, trifluoromethoxy, azido, —NR¹³SO₂R¹⁵, —SO₂NR¹²R¹³, —C(O)R¹², —C(O)OR¹², —OC(O)R¹², —NR¹³C(O)OR¹⁵, —NR¹³C(O)R¹², —C(O)NR¹²R¹³, —NR¹²R¹³, —OR¹², C₁-C₁₀ alkyl, C₂-C₁₀ alkenyl, C₂-C₁₀ alkynyl, —(CR¹³R¹⁴)ₜ(C₆-C₁₀ aryl), and —(CR¹³R¹⁴),(4-10 membered heterocyclic), wherein t is an integer from 0 to 5;
R⁸ is H, —OR¹², —NR¹²R¹³, —NR¹²C(O)R¹³, cyano, —C(O)OR¹³, —SR¹², —(CR¹³R¹⁴),(4-10 membered heterocyclic), wherein t is an integer from 0 to 5, or C₁-C₆ alkyl, wherein said heterocyclic and alkyl moieties are optionally substituted by 1 to 3 R⁶ substituents;
R⁹ is —(CR¹³R¹⁴)ₜ(imidazolyl) wherein t is an integer from 0 to 5 and said imidazolyl moiety is optionally substituted by 1 or 2 R⁶ substituents;
each R¹⁰ and R¹¹ is independently selected from the substituents provided in the definition of R⁶;
each R¹² is independently selected from H, C₁-C₁₀ alkyl, —(CR¹³R¹⁴)ₜ(C₃-C₁₀ cycloalkyl), —(CR¹³R¹⁴)ₜ(C₆-C₁₀ aryl), and —(CR¹³R¹⁴),(4-10 membered heterocyclic), wherein t is an integer from 0 to 5; said cycloalkyl, aryl and heterocyclic R¹² groups are optionally fused to a C6-C₁₀ aryl group, a C₅-C₈ saturated cyclic group, or a 4-10 membered heterocyclic group; and the foregoing R¹² substituents, except H, are optionally substituted by 1 to 3 substituents independently selected from halo, cyano, nitro, trifluoromethyl, trifluoromethoxy, azido, —C(O)R¹³, —C(O)OR¹³, —OC(O)R¹³, —NR¹³C(O)R¹⁴, —C(O)NR¹³R¹⁴, —NR¹³R¹⁴, hydroxy, C₁-C₆ alkyl, and C₁-C₆ alkoxy;
each R¹³ and R¹⁴ is independently H or C₁-C₆ alkyl, and where R¹³ and R¹⁴ are as —(CR¹³R¹⁴)_{q} or (CR¹³R¹⁴), each is independently defined for each iteration of q or t in excess of 1;
R¹⁵ is selected from the substituents provided in the definition of R¹² except R¹⁵ is not H;
R¹⁶ is selected from the list of substituents provided in the definition of R¹² and —SiR¹⁷R¹⁸R¹⁹;
R¹⁷, R¹⁸ and R¹⁹ are each independently selected from the substituents provided in the definition of R¹² except R¹⁷, R¹⁸ and R¹⁹ are not H; and
provided that at least one of R³, R⁴ and R⁵ is —(CR¹³R¹⁴)ₜC≡CR¹⁶ wherein t is an integer from 0 to 5 and R¹³, R¹⁴, and R¹⁶ are as defined above; and
(b) compounds of the formula 2 shown below: 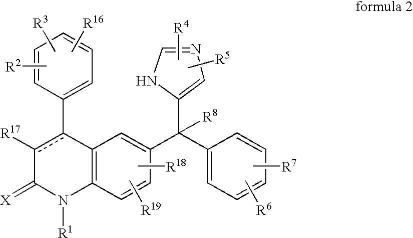
the pharmaceutically acceptable salts, prodrugs and solvates, wherein the dashed line indicates that the bond between C-3 and C-4 is a single or double bond;
X is oxygen or sulfur;
R¹ is hydrogen, C₁-C₁₂alkyl, Ar¹, Ar²C₁-Calkyl, quinolinylC₁-C₆alkyl, pyridylC₁-C₆alkyl,
hydroxyC₁-C₆alkyl, C₁-C₆alkyloxyC₁-C₆alkyl, mono- or di(C₁-C₆alkyl)aminoC₁-C₆alkyl, aminoC₁-C₆alkyl, or a radical of formula -Alk¹—C(═O)—R⁹, -Alk¹—S(O)—R⁹ or -Alk¹—S(O)₂—R⁹;
wherein Alk¹ is C₁-C₆alkanediyl;
R⁹ is hydroxy, C₁-C₆alkyl, C₁-C₆alkyloxy, amino, C₁-C₆alkylamino or C₁-C₈alkylamino substituted with C₁-C₆alkyloxycarbonyl;
R², R³ and R¹⁶ each independently are hydrogen, hydroxy, halo, cyano, C₁-C₆alkyl, C₁-C₆alkyloxy, hydroxyC₁₋₆alkyloxy, C₁-C₆alkyloxyC₁-C₆alkyloxy, aminoC₁-C₆alkyloxy, mono- or di(C₁-C₆alkyl)aminoC₁-C₆alkyloxy, Ar¹, Ar²C₁-C₆alkyl, Ar² oxy, Ar²C₁-C₆alkyloxy, hydroxycarbonyl, C₁-C₆alkyloxycarbonyl, trihalomethyl, trihalomethoxy, C₂-C₆alkenyl, or 4,4-dimethyloxazolyl; or
when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂CH₂—CH₂— (a-5), or
—CH═CH—CH═CH— (a-6);
R⁴ and R⁵ each independently are hydrogen, halo, Ar¹, C₁-C₆alkyl, hydroxyC₁-C₆alkyl, C₁-C₆alkyloxyC₁-C₆alkyl, C₁-C₆alkyloxy, C₁-C₆alkylthio, amino, hydroxycarbonyl, C₁-C₆alkyloxycarbonyl, C₁-C₆alkylS(O)C₁-C₆alkyl or C₁ alkylS(O)₂C₁-C₆alkyl;
R⁶ and R⁷ each independently are hydrogen, halo, cyano, C₁-C₆alkyl, C₁-C₆aikyloxy, Ar²oxy, trihalomethyl, C₁-C₆alkylthio, di(C₁-C₆alkyl)amino, or
when on adjacent positions R⁶ and R⁷ taken together may form a bivalent radical of formula
—O—CH₂—O— (c-1), or
—CH═CH—CH═CH— (c-2);
R⁸ is hydrogen, C₁₋₆alkyl, cyano, hydroxycarbonyl, C₁-C₆alkyloxycarbonyl, C₁-C₆alkylcarbonylC₁-C₆alkyl, cyanoC₁-C₆alkyl, C₁-CealkyloxycarbonylC₁-C₆alkyl, carboxyC₁-C₆alkyl, hydroxyC₁-C₆alkyl, aminoC₁-C₆alkyl, mono- or di(C₁-C₆alkyl)aminoC₁-C₆alkyl, imidazolyl, haloC₁-C₆ alkyl, C₁-C₆alkyloxyC₁-C₆alkyl, aminocarbonylC₁-C₆alkyl, or a radical of formula
—O—R¹⁰ (b-1),
—S—R¹⁰ (b-2),
—N—R¹¹R¹² (b-3),
wherein R¹⁰ is hydrogen, C₁-C₆alkyl, C₁-C₆alkylcarbonyl, Ar¹, Ar²C₁-C₆alkyl, C₁-C₆alkyloxycarbonylC₁-C₆alkyl, or a radical or formula -Alk²-OR¹³ or -Alk²-NR¹⁴R¹⁵
R¹¹ is hydrogen, C₁-C₁₂alkyl, Ar¹ or Ar²C₁-C₆alkyl;
R¹² is hydrogen, C₁-C₆alkyl, C₁-C₁alkylcarbonyl, C₁-C₆alkyloxycarbonyl, C₁-C₆alkylaminocarbonyl, Ar¹, Ar²C₁-C₆alkyl, C₁-C₆alkylcarbonylC₁-C₆alkyl, a natural amino acid, Ar¹ carbonyl, Ar²C₁-C₆alkylcarbonyl, aminocarbonylcarbonyl, C₁-C₆alkyloxyC₁-C₆alkylcarbonyl, hydroxy, C₁-C₆alkyloxy, aminocarbonyl, di(C₁-Calkyl)aminoC₁-C₆alkylcarbonyl, amino, C₁-C₆alkylamino, C₁-C₆alkylcarbonylamino, or a radical of formula -Alk²—OR¹³ or -Alk²—NR¹⁴R¹⁵; wherein
Alk² is C₁-C₆alkanediyl;
R¹³ is hydrogen, C₁-C₆alkyl, C₁-C₆alkylcarbonyl, hydroxyC₁-C₆alkyl, Ar¹ or Ar²C₁-C₆alkyl;
R¹⁴ is hydrogen, C₁-C₆alkyl, Ar¹ or Ar²C₁-C₆alkyl;
R¹⁵ is hydrogen, C₁-C₆alkyl, C₁-C₆alkylcarbonyl, Ar¹ or Ar²C₁-C₆alkyl;
R¹⁷ is hydrogen, halo, cyano, C₁-C₆alkyl, C₁-C₆alkyloxycarbonyl, Ar¹;
R¹⁸ is hydrogen, C₁-C₆alkyl, C₁-C₆alkyloxy or halo;
R¹⁹ is hydrogen or C₁-C₆alkyl;
Ar¹ is phenyl or phenyl substituted with C₁-C₆alkyl, hydroxy, amino, C₁-C₆alkyloxy or halo; and
Ar² is phenyl or phenyl substituted with C₁-C₆alkyl, hydroxy, amino, C₁-C₆alkyloxy or halo.

2. The pharmaceutical composition according to claim 1, wherein said FTase inhibitor is a compound of formula 1, wherein RI is H, C₁-C₆ alkyl, or cyclopropylmethyl; R² is H; R³ is —C≡CR¹⁶; and R⁸ is —NR¹²R¹³, —OR¹², or a heterocyclic group selected from triazolyl, imidazolyl, pyrazolyl, and piperidinyl, wherein said heterocyclic group is optionally substituted by an R⁶ group.

3. The pharmaceutical composition according to claim 2, wherein R⁹ of the compound of formula 1 is imidazolyl optionally substituted by C₁-C₆ alkyl; R³ is hydroxy, amino, or triazolyl; and R⁴, R⁵, R¹⁰ and R¹¹ are each independently selected from H and halo.

4. The pharmaceutical composition according to claim 1, wherein said FTase inhibitor is a compound of formula 1, wherein R¹ of the compound of formula 1 is —(CR¹³R¹⁴)ₜ(C₃-C₁₀ cycloalkyl) wherein t is an integer from 0 to 3; R² is H; and R⁸ is —NR¹²R¹³, —OR¹², or a heterocyclic group selected from triazolyl, imidazolyl, pyrazolyl, and piperidinyl, wherein said heterocyclic group is optionally substituted by an R⁶ group.

5. The pharmaceutical composition according to claim 4, wherein R⁹ of the compound of formula 1 is imidazolyl optionally substituted by C₁-C₆ alkyl; R⁸ is hydroxy, amino, or triazolyl; R³ is —C≡CR¹⁶; R⁴, R⁵, R¹⁰ and R¹¹ are each independently selected from H and halo; and R¹ is cyclopropylmethyl.

6. The pharmaceutical composition according to claim 5, wherein R³ of the compound of formula 1 is ethynyl.

7. The pharmaceutical composition according to claim 2, wherein R³ of the compound of formula 1 is ethynyl.

8. The pharmaceutical composition according to claim 1, wherein the FTase inhibitor is selected from the group consisting of:
6-[(4-Chloro-phenyl)-hydroxy-(3-methyl-3H-imidazol-4-yl)-methyl]-4-(3-ethynyl-phenyl)-1-methyl-1H-quinolin-2-one (enantiomer A);
6-[(4-Chloro-phenyl)-hydroxy-(3-methyl-3H-imidazol-4-yl)-methyi]-4-(3-ethynyl-phenyl)-1-methyl-1H-quinolin-2-one (enantiomer B);
6-[Amino-(4-chloro-phenyl)-(3-methyl-3H-imidazol-4-yl)-methyl]-4-(3-ethynyl-phenyl)-1-methyl-1-H-quinolin-2-one (enantiomer A);
6-[Amino-(4-chloro-phenyl)-(3-methyl-3H-imidazol-4-yl)-methyl]-4-(3-ethynyl-phenyl)-1methyl-1H-quinolin-2-one (enantiomer B);
6-[(4-Chloro-phenyl)-hydroxy-(3-methyl-3H-imidazol-4-yl)-methyl]-4-(3-ethynyl-4-fluoro-phenyl)-1-methyl-1-H-quinolin-2-one;
and the pharmaceutically acceptable salts, prodrugs and solvates of the foregoing compounds.

9. The pharmaceutical composition according to claim 1, wherein said FTase inhibitor is a compound of formula 2, wherein X is oxygen.

10. The pharmaceutical composition according to claim 9 wherein the dotted line of the compound of formula 2 is a bond.

11. The pharmaceutical composition according to claim 10 wherein R¹ of formula 2 is hydrogen, C₁-C₆alkyl, C₁-C₆alkyloxyC₁-C₆alkyl, di(C₁-C₆alkyl)aminoC₁-C₆alkyl.

12. The pharmaceutical composition according to claim 11, wherein R³ is hydrogen or halo; and R² is halo, C₁-C₆alkyl, C₂-C₆alkenyl, C₁-C₆alkyloxy, trihalomethoxy or hydroxyC₁-C₆alkyloxy.

13. The pharmaceutical composition according to claim 12, wherein R⁸ is hydrogen, hydroxy, haloC₁-C₆alkyl, hydroxyC₁-C₆alkyl, cyanoC₁-C₆alkyl, C₁-C₆alkyloxycarbonylC₁-C₆alkyl, imidazolyl, or a radical of formula —NR¹¹R¹² wherein R¹¹ is hydrogen or C₁-C₁₂alkyl and R¹² is hydrogen, C₁-C₆alkyl, C₁-C₆alkyloxy, hydroxy, C₁-C₆alkyloxyC₁-C₆alkylcarbonyl, or a radical of formula -Alk²—OR¹³ wherein R¹³ is hydrogen or C₁-C₆alkyl.

14. The pharmaceutical composition according to claim 1 wherein the FTase inhibitor is selected from the group consisting of:
4-(3-chlorophenyl)-6-[(4-chlorophenyl)hydroxy(1-methyl-1H-imidazol-5-yl)methyl]-1-methyl-2(1H)-quinolinone,
6-[amino(4-chlorophenyl)(1-methyl-1H-imidazol-5-yl)methyl]-4-(3-chlorophenyl)-1-methyl-2(1H)-quinolinone (enantiomer A);
6-[amino(4-chlorophenyl)(1-methyl-1-H-imidazol-5-yl)methyl]-4-(3-chlorophenyl)-1-methyl-2(1H)-quinolinone (enantiomer B);
6-[(4-chlorophenyl)hydroxy(1-methyl-1-H-imidazol-5-yl)methyl]-4-(3-ethoxyphenyl)-1-methyl-2(1H)-quinolinone;
6-[amino(4-chlorophenyl)(1-methyl-1H-imidazol-5-yl)methyl]-4-(3-ethoxyphenyl)-1methyl-2(1H)-quinolinone,
6-amino(4-chlorophenyl)(1-methyl-1H-imidazol-5-yl)methyl]-1-methyl-4-(3-propylphenyl)-2(1H)-quinolinone; and the pharmaceutically acceptable salts, prodrugs and solvates of the foregoing compounds.

15. The pharmaceutical composition according to claim 1, wherein the HMG CoA reductase inhibitor is selected from the group consisting of atorvastatin, pravastatin, lovastatin, compactin, fluvastatin, simvastatin, and ZD4522 (AstraZeneca) and the pharmaceutically acceptable salts of the foregoing compounds.

16. The pharmaceutical composition according to claim 15, wherein the HMG CoA reductase inhibitor is selected from the group consisting of atorvastatin, lovastatin, pravastatin and simvastatin and the pharmaceutically acceptable salts of the foregoing compounds.

17. The pharmaceutical composition according to claim 16, wherein the HMG CoA reductase inhibitor is selected from the group consisting of atorvastatin and lovastatin and the pharmaceutically acceptable salts of the foregoing compounds.

18. The pharmaceutical composition according to claim 17, wherein the HMG CoA reductase inhibitor is atorvastatin and its pharmaceutically acceptable salts.

19. The pharmaceutical composition according to claim 1, wherein said abnormal cell is cancer or a benign proliferative disorder.

20. A method of treating cancer or a benign proliferative disorder in a mammal, comprising administering to said mammal an effective amount of a pharmaceutical composition according to claim 1.
